(19) United States
(12) Patent Application Publication
(10) Pub. No.: US 2018/0345247 A1
(43) Pub. Date: Dec. 6, 2018

(54) MODIFICATION OF HALLOYSITE MINERAL ADSORBENT BY DENDRITIC POLYMER IN CONVERGENT SYNTHETIC ROUTE AND ITS APPLICATION

(71) Applicant: Somaye Akbari, Woodbridge (CA)

(72) Inventor: Somaye Akbari, Woodbridge (CA)

(21) Appl. No.: 15/615,785

(22) Filed: Jun. 6, 2017

(51) Int. Cl. B01J 20/26; A61K 47/24; C08G 83/00; B01J 20/28; C02F 1/28; B01D 69/14

(52) U.S. Cl. CPC B01J 20/265; A61K 47/24; C08G 83/004; B01J 20/28033; C02F 2101/308; B01D 69/147; C02F 1/281; C02F 1/285; C02F 1/288

(57) ABSTRACT

The method relates to the modification of mineral adsorbent particularly halloysite in the form of clay or tube using dendritic polymer for treating wastewater containing ionic or nonionic water pollutants such as heavy metal ions, dyes, surfactants, high molecular weight coagulants and mineral oils. The method will increase surface activity of the adsorbent and can be applied to create positive or negative charges on the surface of the adsorbent. The modified mineral can be used as: adsorbent of pollutant such as dye, heavy metal ion, aromatic material from aqueous solutions, for removal of cations from aqueous solutions, for removal of anions from aqueous solutions, as filler in nano-composite, as nano-particle in polymeric membrane, adsorbent for soil, and special pharmaceutical application.

### REFERENCES CITED

CN 102492173 B Halloysite with modified surface and preparation method for halloysite
CN 103087553 A Maleic anhydride grafted modified halloysite nanotube and preparation method thereof
CN 104119704 A Surface modification treatment method of halloysite nanotube
CN 103923497 A Preparation method of halloysite modified bentonite material
U.S. Pat. No. 6,080,319 A Chemical methods for removing contaminants from water
CN 103509161 A Preparation method of hydrophilic magnetic halloysite surface molecularly-imprinted nano composite material
CN 102343101A Preparation method and application of functionalized kaolin nanotube-antisense oligonucleotide medicine carrier
Qu, Rongjun, et al. “Syntheses, characterization, and adsorption properties for metal ions of silica-gel functionalized by ester- and amino-terminated dendrimer-like polyamidoamine polymer.” Microporous and mesoporous materials 97.1 (2006): 58-65.

### BACKGROUND OF THE INVENTION

Industrial wastewaters usually contain substances that are severely harmful to the environment. For example, textile dyeing is a chemically intensive process which consumes large quantities of water. In a typical dyeing process, 50-100% of dye is fixed on the fiber, and the unfixed dyes are discharged into the dye-baths or into the wastewater from the subsequent textile-washing operations. To comply with stringent discharge limits, multi-step and complicated treatment is usually required. Among different methods available for dye removal or other contaminant, adsorption is a highly efficient and low-cost process. Numerous types of adsorbent such as activated carbon, silica, zeolite, kaolinite, bentonite, etc. have been employed for the adsorption techniques, but in some cases the adsorbents are prohibitively expensive or in other cases they are not suitable for the application. As a result, there's a need to continue to develop and identify cheaper adsorbent materials with higher performance but a lower cost.

Halloysite is natural-mineral clay with hollow nanotubular structure. It is abundant in many countries such as China, the United States, Brazil and France. As a result, they are one of the cheapest existing adsorbents in many regions of the world. Halloysite structure has collected notable properties in material science due to its special features of large surface area, unique tubular structure, high porosity, high length-to-diameter ratio, and high receptor surface chemistry, which have made this nanomaterial to be utilized as an adsorbent material. This substance naturally contains aluminosilicate nanotubes with a 1:1 Al:Si ratio and a stoichiometry of Al₂Si₂O₅(OH)₄.nH₂O. Halloysite consist of octahedral sheet of Al—OH groups on the internal surface and siloxane groups (Si—O—Si) on the external surface. These variances have led to a negatively charged outer surface and a positively charged inner lumen at pH range of 2 to 8.

Modification of phyllosilicates clay minerals, especially halloysite and kaolinite to improve capacity or enhance the activity of these as adsorbent, is subject of numerous patents. The first step typically involves washing off the powder to remove impurities such as iron, which often mask the active sites, and then followed by activation with coupling agent. The common coupling agents are from silane coupling agent group. Silane coupling agents are used attaching organic components to inorganic base material. The activation sites are terminated by adding an organic component such as amines. This step is the distinctive difference between most of the patents.

Patent “CN 102492173 B” uses catalysts for aminolysis ester groups. At first, ester group is activated, and then followed by aminolysis. Patent “CN 103087553 A” introduces an active amino group onto surface of halloysite nanotubes which is already activated by silane coupling agent. Surface modification terminated by grafting maleic anhydride on the surface of the halloysite nanotubes. Patent “CN 104119704 A” teaches the modification of halloysite nanotubes with silane group by adding epoxy resins to produce a nanocomposite material. This patent uses halloysite nanotubes to improve mechanical and thermal properties of epoxy resins. Patent “CN 103923497 A” uses a unique chemistry to combine halloysite with bentonite. The new clay will have the special features and properties of both ingredients and can be used in numerous applications. Patent “CN 102343101A” describes a method to functionalize kaolin nanotube-antisense oligonucleotide as a medicine carrier. For silane coupling agent, 3-aminopropyl triethoxysilane was selected and the functionalized group was created by reacting with antisense oligonucleotides. The product is claimed to be a stable function of halloysite nanotube antisense oligonucleotide solution. Patent “CN 103509161 A” recommended the use of aminopropyttriethoxysilane (APTES) for silane coupling agent. Surface amination was carefully controlled by surface atom transfer radical polymerization and the final product was a composite material. Patent “U.S. Pat. No. 6,080,319 A” teaches the use of organoclay with naturally positive or negative charge on the surface as a sorbent. However, this patent does not advocate the use of silane as coupling agent, but instead modification is performed with at least one of an organic ionic compound in stoichiometric excess and an amphoteric surfactant. Qu, R., et al. (2006) investigated divergent synthetic route to modify silica till generation 4 for adsorption of metal ion. The long-time procedure for synthetic route is limitation of their investigation which it improved with convergent synthetic route in our patent.

### BRIEF DESCRIPTION OF DRAWINGS

Scheme 1: Halloysite-NH₂

Scheme 2: Halloysite-amino terminated dendritic polymer via divergent synthetic route

Scheme 3: Halloysite-carboxylic acid terminated group

Scheme 4: Halloysite-amino terminated dendritic polymer via convergent synthetic route with COOH activation

Scheme 5: Halloysite-amino terminated dendritic polymer via convergent synthetic route with MA activation

Scheme 6: Halloysite-amino terminated dendritic polymer

FIG. 1: Dye Removal (%) of raw HNT, HNT_{G1}, HNT_{G2} and HNT_{G3} in divergent modified halloysite.

FIG. 2: Dye Removal (%) of raw HNT, HNT_{PPIG2}, HNT_{PPIG5}, HNT_{PAMAMG2} and HNT_{PAMAMG4} in convergent modified halloysite.

FIG. 3: Fe²⁺ Removal (%) of raw HNT and HNT_{G1} at different times, (adsorbent dosage: 0.6 g/L, pH: 6).

FIG. 4: Fe²⁺ Removal (%) of different generation of HNT modified by divergent method, (adsorbent dosage: 0.6 g/L, pH: 6).

FIG. 5: Fe²⁺ Removal (%) of HNT_{G1} at different dosages, (time: 10 min, pH: 6).

FIG. 6: The dye removal for raw silica, silica APTES and silica-G1 till 15 min.

FIG. 7. Removal of Cr (III) by HNT-dendrimer, HNT-COOH and HNT-impure (Adsorbent dosage: 0.4 g/L).

FIG. 8 The dye removal for modified HNT in convergent synthetic route at pH 3 and 5.

### SUMMARY OF THE INVENTION

The new approach in the modification of halloysite using dendritic polymer includes dendrimer as well as hyperbranched polymer. Dendritic polymers adsorb contaminant from wastewater in two ways: binding with the abundant functional end groups, and encapsulating in the interior between the branches. To introduce amine-terminated dendritic polymer on the surface of halloysite, aminosilane agents such as (3-Aminopropyl) triethoxysilane (APTES) are used to functionalize the halloysite in the process of silanization. In this case, amino groups are introduced on the surface of halloysite, which could act as the core for the synthesis of the dendritic structure on the halloysite via divergent or convergent synthetic route.

Modification Method Via Divergent and Convergent Dendritic Synthetic Route: Modification in Divergent Synthetic Route:

1. The first modification step to modify is changed all hydroxyl groups to amine group by (3-Aminepropyl) triethoxysilane (APTES).

2. In next step, amine dendritic groups are added via divergent synthesis from zero to n^{th} generation by repeating the Michael addition of methyl acrylate and amidation of the esters groups with ethylenediamine. Scheme 2 shows first Michael addition of methyl acrylate to reach Generation G0.5 followed by amidation of the esters groups to terminate to Generation G1. By following the same Michael addition of methyl acrylate followed by amidation of the esters groups Generation G1.5 and G2 can be reached which is also presented in Scheme 2. By repeating the same steps G2.5 and G3 will be produced which has more active sites compare to previous generations. The reaction and termination could be continued to create generation n^{th} of the modified HNTs. It worth to note that either or both methyl acrylate and ester group could be replace with equivalent components in any generation. It seems that third generation could be the sufficient modification for most applications.

Modification in Convergent Synthetic Route:

Four different strategy could be applied in convergent synthetic route:

1. Surface modification by convergent method for halloysite nanotube (HNT), compromising steps of: (1) purification of HNTs with hydrochloric acid solution followed by water washing and drying; (2) introducing amino group on surface of the acidified HNT by adding silane coupling agent solution; (3) adding carboxylic acid terminated dendritic groups via convergent synthesis.

2. Surface modification by convergent method for halloysite nanotube (HNT), compromising steps of: (1) purification of HNTs with hydrochloric acid solution followed by water washing and drying; (2) introducing amino group on surface of the acidified HNT by adding silane coupling agent solution; (3) adding amine dendritic groups by the Michael addition of methyl acrylate (MA), (4) adding amino terminated dendritic polymer via convergent synthesis.

3. Surface modification by convergent method for halloysite nanotube (HNT), compromising steps of (1) purification of HNTs with hydrochloric acid solution followed by water washing and drying; (2) converting all hydroxyl group to carboxylic acid group by adding a dicarboxylic acid; (3) adding amine terminated dendrimer to carboxylic acid group.

4. Surface modification by convergent method for halloysite nanotube (HNT), compromising steps of: (1) purification of HNTs with hydrochloric acid solution followed by water washing and drying; (2) introducing amino group on surface of the acidified HNT by adding silane coupling agent solution; (3) converting all amine group to carboxylic acid group by adding a dicarboxylic acid; (4) adding amino terminated dendritic polymer via convergent synthesis.

In third and fourth convergent synthetic route all hydroxyl and amino groups are first converted to carboxylic acid groups with different type of carboxylic acid. The list of carboxylic acid is listed in Table 1.

TABLE 1
The list of carboxylic acid to produce halloysite-COOH terminal group

| | | Molecular | |
|---|---|---|---|
| | | Weight | Molecular |
| Name | Chemical structure | (g/mol) | formula |
| Phenyl- phosphonic acid | 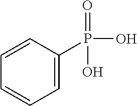 | 158.09 | C₆H₇O₃P |
| Glutaric acid | 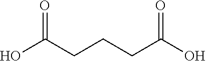 | 132.11 | C₅H₈O₄ |
| Malonic acid | 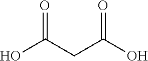 | 104.06 | C₃H₄O₄ |
| Oxalic acid | 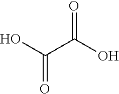 | 90.03 | C₂H₂O₄ |
| Succinic anhydride | 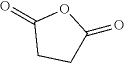 | 100.07 | C₄H₄O₃ |

The chemical reaction is illustrated in Scheme 3.

The application of the functionalized halloysite could be effective for removal of heavy metal cations from the soil or aqueous solution.

Furthermore, this modification could be utilized for convergent synthetic route in dendritic strategy as revealed in Scheme 4.

The final dendritic structure on functionalized halloysite is illustrated in Scheme 6.

### Example 1

Surface Charge: Zeta Potential of Halloysite Nanotube

The zeta potential values of different samples are reported in Table 2. These results indicate that the surface of raw halloysite is negatively charged and as the generation of the amine dendritic structure on halloysite increases, the overall surface charge increases up to +25.30 (mV) for generation three (HNT_{G3}). The modified halloysite through convergent method also showed the same behaviour and the grafting of different generations of PPI and PAMAM to halloysite also resulted in more positive surface, which strongly confirms the existence of amine groups in the structure of the modified halloysite. These negative and positive charges are useful for adsorption of cationic or anionic contaminant, respectively.

TABLE 2
Zeta potential of HNT samples

| Sample | Zeta potential |
|---|---|
| raw halloysite | −34.50 |
| H_{APTES} | −13.30 |
| HNT_{G1} | +1.85 |
| HNT_{G2} | +20.40 |
| HNT_{G3} | +25.30 |
| HNT_{PPIG2} | +6.29 |
| HNT_{PPIG5} | +23.30 |
| HNT_{PAMAMG2} | +1.83 |
| H_{PAMAMG4} | −2.50 |

Surface Charge: Zeta Potential of Silica

The zeta potential values of different silica samples and modified ones are reported in Table 3. Silica G1 is modified in divergent synthetic route and silica-convergent is modified according to Claim 2.

TABLE 3
Zeta potential of Silica samples

| Sample | Zeta potential |
|---|---|
| raw silica | −2.99 |
| silica APTES | −5.16 |
| silica G1 | +13.3 |
| Silica-convergent | −0.339 |

### Example 2

Acid Dye Removal for HNT

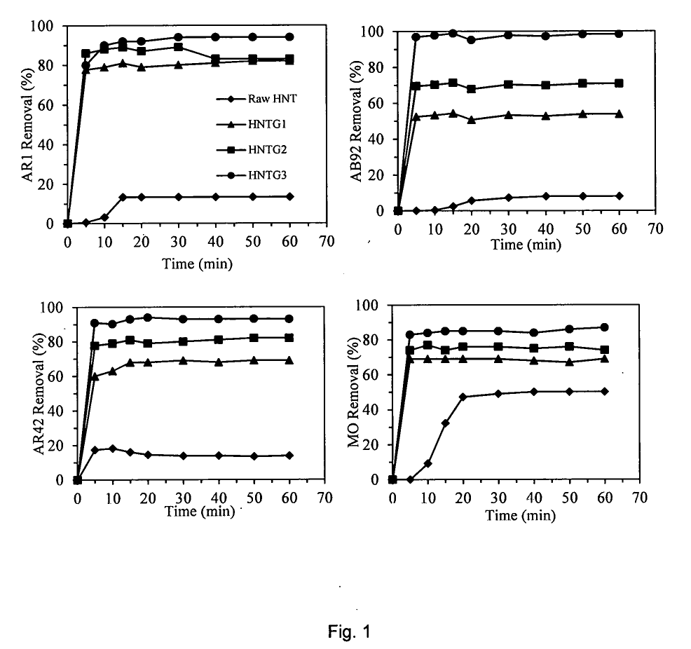

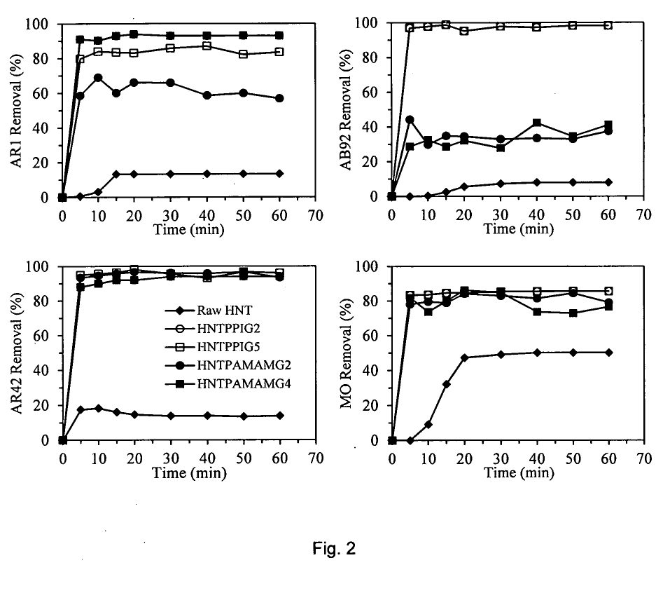

The removal efficiencies of C.I. Acid Red 1 (AR1), C.I. Acid Red 42 (AR42), Acid Blue 92 (AB92) and Methyl orange (MO) AR1, AR42, AB92 and MO by halloysite modified via divergent and convergent methods are shown in FIGS. 1 and 2. It is evident from FIG. 1 that the dye removal of raw halloysite is less than 50%, but the removal efficiency increases after synthesis of each generation and reaches to the maximum of 98.2% for AB92 by HNT_{G3}.

The improvement in dye removal (%) after grafting of dendritic structures on the halloysite can be seen in FIG. 2. Higher removal percentages are obtained by hyperbranched modified halloysite rather than dendrimer modified halloysite. Also, the overall efficiency of divergent method is higher, than convergent; however, the modification steps of convergent method are more facile, shorter and easier to perform.

### Example 3

Heavy Metal Ion Adsorption for HNT

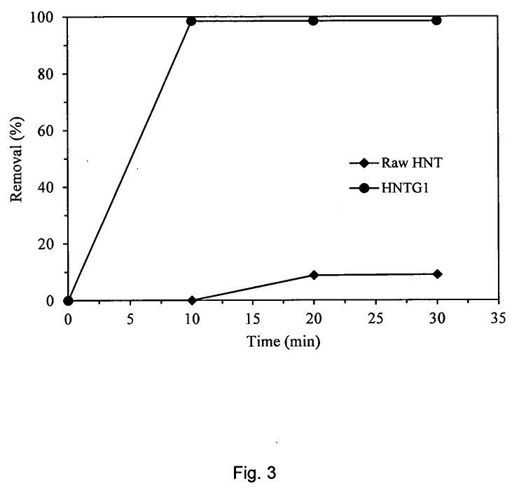

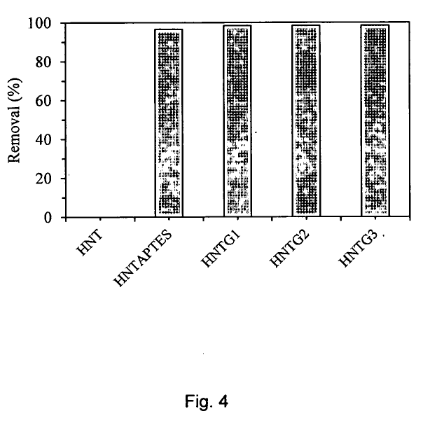

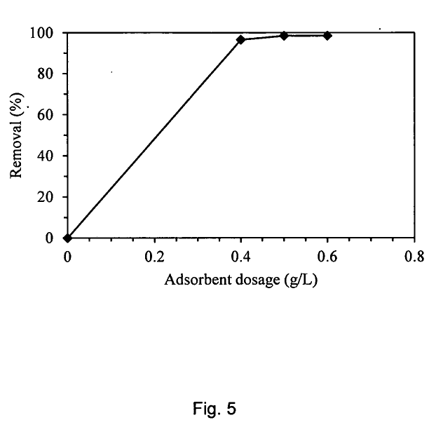

The effect of time, dendrimer generation, and adsorbent dosage on Fe²⁺ removal using the amine terminated halloysite modified by divergent route are respectively represented in FIGS. 3, 4, and 5. The results clearly indicate that the removal (%) increases significantly from 9.02% to 98.48% after the synthesis of first generation of amine dendritic structure, and with higher generation polymer, the heavy metal ion removal remains relatively unchanged. Since the efficiency reaches its maximum value after only 10 min of adsorption process, this specific reaction or contact time has been selected as the optimum time in the removal procedure.

### Example 4

Acid Dye Removal for Silica

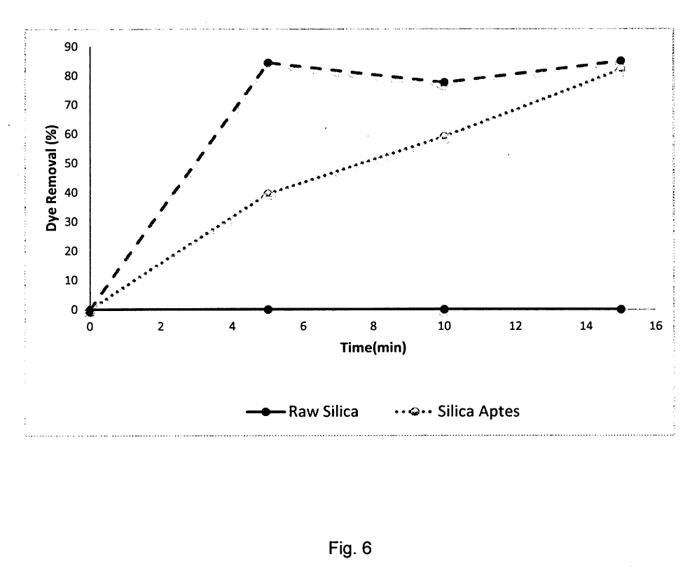

Surface modification by divergent method for silica compromising steps of: (1) purification of silica with hydrochloric acid solution followed by water washing and drying; (2) introducing amino group on surface of the acidified silica by adding silane coupling agent solution (Silica APTES); (3) adding amine dendritic groups via divergent synthesis by the Michael addition of methyl acrylate (MA) and amidation of the esters groups with ethylene diamine (ED) (Silica G1). FIG. 6 reveal the dye removal for raw silica, silica APTES and silica-G1 till 15 min.

### Example 5

Heavy Metal Ion Adsorption for HNT in Convergent Method by Carboxylic Acid Linkage

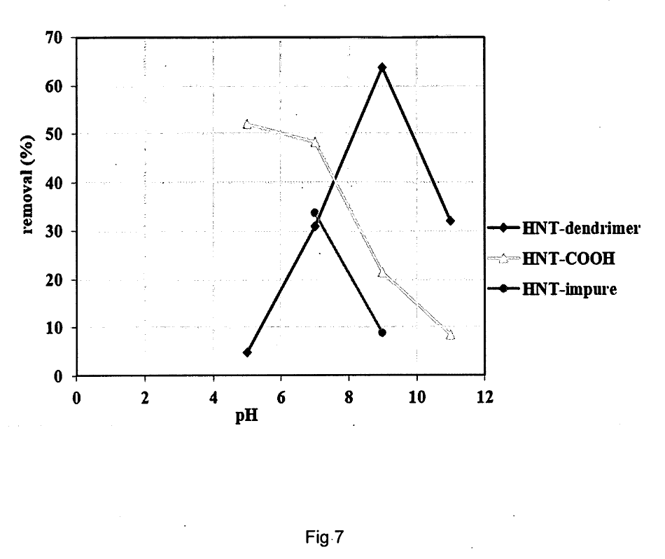

One of the most important parameters affecting the adsorption capacity is the pH adsorption. The effect of pH on the adsorption of Cr (III) shown in FIG. 7. The maximum Cr (III) adsorption occurred at pH=9. It can be seen in FIG. 7 that dendrimer functionalization of HNT has improved the removal efficiency for about 30%. At pH=5, the soluble complex CrOH²⁺ becomes the major component, also at this pH positively charged (—NH₃⁺) surface of dendrimer cause repulsion between metal ion and adsorbent, as result, adsorption efficiency decreases. Between pH 7 to 10 the total of Cr (III) is in the form of neutral Cr (OH)₃ (aq). As the pH of system increases, positively charge sites of dendrimer decreased, so, efficient adsorption increases. Also, the capacity of HNT-dendrimer was found to be 67.5 mg/g.

### Example 6

Acid Dye Adsorption for HNT in Convergent Method by Carboxylic Acid Linkage

Carboxylic acid group were synthesized into the halloysite nano tube (HNT); then amine-terminated dendritic hyperbranched polymer with convergent method reacted with carboxylic acid functionalized halloysite. Raw halloysite were washed with HCL and functionalized with 3-amino poropyltriethoxysilane (APTES), HNT-NH₂ was reacted with succinic anhydride in order to make carboxylic acid group in to the structure. Then amine terminated group added to HNT-COOH in convergent method.

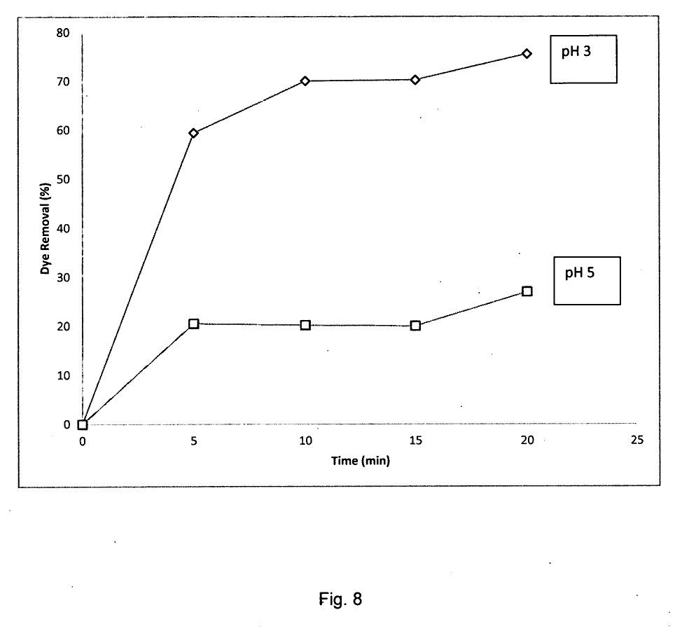

Adsorption capacity was investigated by new synthesis adsorption. The optimum adsorbent dosage and pH for dye removal of CI ACID RED 1 were obtained to be 0.5 g/l and 3, respectively. The dye removal for modified HNT in convergent synthetic route at different pH reveal in FIG. 8.

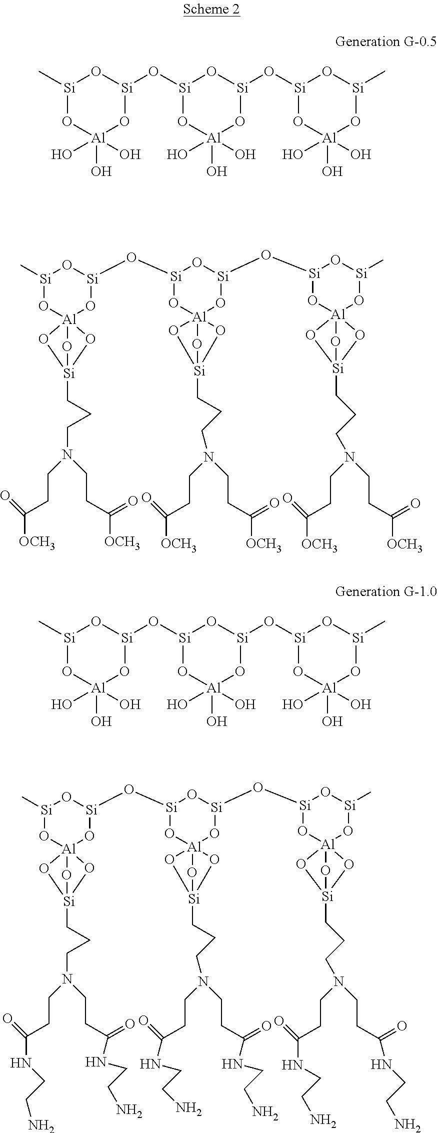

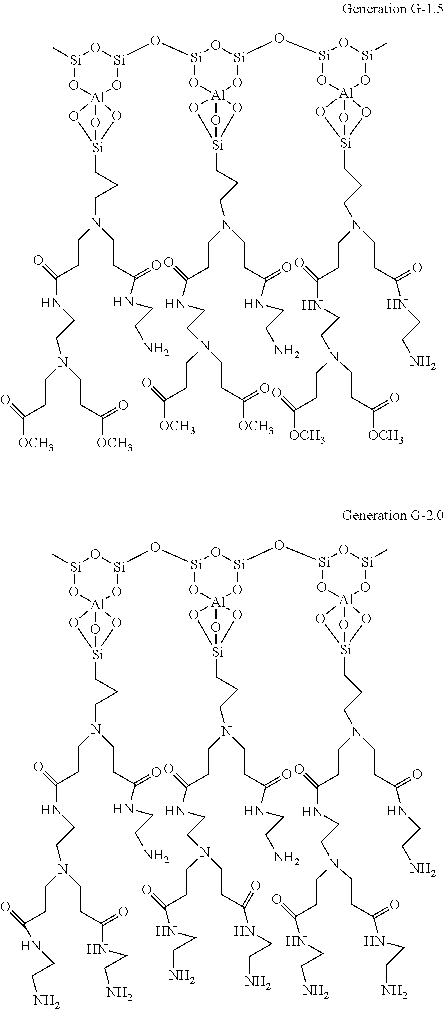

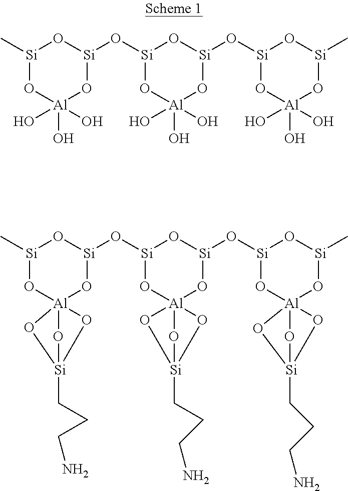

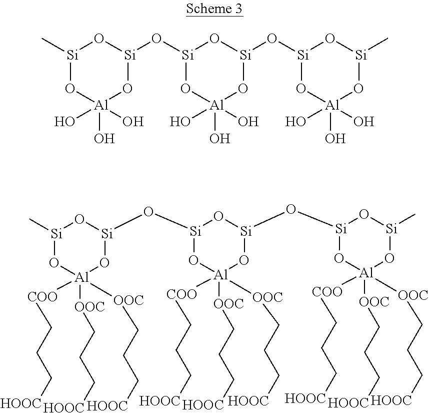

1. Surface modification by convergent method for halloysite nanotube (HNT), compromising steps of: (1) purification of HNTs with hydrochloric acid solution followed by water washing and drying; (2) introducing amino group on surface of the acidified HNT by adding silane coupling agent solution; (3) adding carboxylic acid terminated dendritic groups via convergent synthesis.

2. Surface modification by convergent method for halloysite nanotube (HNT), compromising steps of: (1) purification of HNTs with hydrochloric acid solution followed by water washing and drying; (2) introducing amino group on surface of the acidified HNT by adding silane coupling agent solution; (3) adding amine dendritic groups by the Michael addition of methyl acrylate (MA), (4) adding amino terminated dendritic polymer via convergent synthesis.

3. Surface modification by convergent method for halloysite nanotube (HNT), compromising steps of: (1) purification of HNTs with hydrochloric acid solution followed by water washing and drying; (2) introducing amino group on surface of the acidified HNT by adding silane coupling agent solution; (3) converting all amine group to carboxylic acid group by adding a dicarboxylic acid; (4) adding amino terminated dendritic polymer via convergent synthesis.

4. The modification of claim 3 wherein the dicarboxylic acid group are synthesized without silane coupling agent and directly reacted by hydroxyl group on the raw halloysite.

5. The modification of claim 1 or 2 or 3 wherein the silane coupling agent comprises (3-Aminepropyl)-triethoxysilane (APTES) or (3-aminopropyl)-diethoxy-methylsilane (APDEMS) or (3-aminopropyl)-dimethyl-ethoxysilane (APDMES) or (3-aminopropyl)-trimethoxysilane (APTMS).

6. The modification of claim 2 or 3 or 4 wherein amino group is all amine terminated dendritic group including hyperbranched, dendrigraft and dendrimer polymers.

7. The modification of claim 1 or 6 wherein dendrimer comprises any amine terminated polymer such as PEI (polyethyleneimine), and PVA (polyvinylamine); as well as, any amine terminated dendritic polymer such as PAMAM (polyamidoamine) or PPI (polypropyleneimine) dendrimer; or any hyper-branched amine-terminated polymer.

8. The modification of claim 3 or 4 wherein carboxylic acid comprises phenylphosphonic acid, glutaric acid, malonic acid, oxalic acid, succinic anhydride and poly acrylic acid.

9. The modification of claim 2 wherein half generation could be replaced by generation 1.5 and 2.5 in divergent synthetic route.

10. The modification of claim 2 wherein half generation by divergent method for halloysite nanotube (HNT), compromising steps of: (1) purification of HNTs with hydrochloric acid solution followed by water washing and drying; (2) introducing amino group on surface of the acidified HNT by adding silane coupling agent solution; (3) adding amine dendritic groups via divergent synthesis from zero to nth generation by repeating the Michael addition of methyl acrylate (MA).

11. Surface modified halloysite nanotubes (HNT) of claim 7, 8, 9 or 10 are used as adsorbents for removal of pollutants such as dye, heavy metal ion, aromatic material from aqueous solutions, cations and anions from aqueous solutions.

12. Surface modified halloysite nanotubes (HNT) of claim 7, 8, 9 or 10 are used as filler in nano-composite materials.

13. Surface modified halloysite nanotubes (HNT) of claim 7, 8, 9 or 10 are used as nano-particle in polymeric TFC (thin-film composite) membranes such as forward osmosis, reverse osmosis, nanofiltration, and gas separation membranes.

14. Surface modified halloysite nanotubes (HNT) of claim 7, 8, 9 or 10 are used in bio-nanocomposite and special pharmaceutical application such as biodegradable coating for controlled drug delivery.

15. The modification of claim 7, 8, 9 or 10 wherein the halloysite nanotube is replaced by other mineral adsorbent such as kaolinite, silica, titanium dioxide, graphene oxide, montmorillonite, bentonite or similar.
